(19) United States
(12) Patent Application Publication
(10) Pub. No.: US 2026/0000543 A1
(43) Pub. Date: Jan. 1, 2026

(54) Method and Apparatus for Treating Topical Wounds

(71) Applicant: Starch Medical, Inc., San Jose, CA (US)

(72) Inventors: Xin Ji, San Jose, CA (US); Xueshen Shi, San Jose, CA (US); Xiaoguang Zhang, Beijing (CN)

(21) Appl. No.: 18/759,697

(22) Filed: Jun. 28, 2024

(51) Int. Cl. A61F 13/00

(52) U.S. Cl. CPC A61F 13/00063; A61F 13/0266; A61F 2013/00285

(57) ABSTRACT

A bandage for delivering a powder composition to tissue on a body surface includes a biocompatible powder composition used in combination with a water dissolvable membrane covered by a removable film to provide a safe and effective method of applying topical medications to tissue surfaces and, more specifically, to treating wounds.

### FIELD

The present specification generally relates to a topical hemostat for wound tissue treatment, and more specifically, to a method that uses biocompatible materials in a powder form to achieve hemostasis as well as treatment of tissue on a body surface.

### BACKGROUND

Bleeding wounds may occur as a result of both surgical operations and trauma and can bring a risk of excess blood loss. In such cases, it may be necessary to apply hemostat to control bleeding in a timely manner. It is common to apply biocompatible, hemostatic agents directly to bleeding wound sites to achieve hemostasis (cessation of bleeding) for surgical procedures, trauma treatment and home self-rescue. Products commonly used for treating wounds include hemostats, such as hemostatic powders and dressings including bandages, hemostatic sponges, patches, and gauze.

Hemostats such as powders, sponges, or gauze are useful to control the bleeding if it is mild to moderate. Clinically, it may be required to apply a therapeutic material of a flowable powder format directly to the wound or designated tissue on the skin surface of a human or animal. Powders are also used to manage wounds' moist environments and exudates. Powder format medicines are frequently used to treat skin surfaces for cuts, injuries, ulcers, rashes, and other forms of skin irregularities including corns.

Medicaments in powder format can be applied using a multi-step process that includes first applying powder to the tissue followed by applying a dressing material on top of the powder. This multi-step process is fraught with challenges, however, including powder wastage, difficulty of application, and can be unduly laborious.

There is therefore a need for improved dressing systems with pre-applied medicine, in a flowable powder-format material for direct delivery to the wound or a designated tissue of a body surface, for ease of application, and for effective treatment of wounds and diseases. Further, the product should be convenient to use and capable of being applied topically on an outer bleeding wound surface.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a bandage adapted to deliver a powder composition to tissue on a body surface, comprising: a removable substrate comprising an adhesive on a first surface adapted to face the tissue; a loop having a thickness and defining a recess characterized by a depth, wherein the recess is positioned centrally on said first surface; the powder composition positioned in the recess; a water-soluble membrane adapted to cover the recess and the powder composition positioned in the recess, wherein the water-soluble membrane covers the loop and is adapted to adhere to the first surface of the removable substrate; and a release film adapted to cover the water-soluble membrane and adapted to adhere to the first surface of the removable substrate.

Optionally, the water-soluble membrane is adapted to dissolve in less than 30 seconds after removing the release film and adhering the removable substrate to the tissue.

Optionally, the water-soluble membrane is adapted to dissolve in less than 10 seconds when the water-soluble membrane is in contact with at least 0.05 milliliters of a fluid.

Optionally, the water-soluble membrane has a tensile strength of at least 1N/15 mm.

Optionally, the water-soluble membrane is at least one of non-allergic, non-irritating, or non-cytotoxic.

Optionally, the water-soluble membrane has a thickness between 1 μm and 500 μm.

Optionally, the water-soluble membrane comprises at least one of potato starch, modified starch, hydroxyethyl starch, carboxymethyl cellulose, or microcrystalline cellulose.

Optionally, the water-soluble membrane has a folding endurance between 10 and 1000.

Optionally, the adhesive is at least one of a pressure sensitive adhesive or an acrylic based adhesive.

Optionally, the removable substrate comprises at least one of polyethylene (PE), polyphenylene ether (PPE), polyvinyl chloride (PVC), cotton, non-woven fiber, synthetic fabric, an elastic fabric, a water-proof material, or a silicone gel material.

Optionally, the loop comprises at least one of a silicone gel, foam, or cotton and is of a rectangular shape, a square shape, an oval shape, or a circular shape.

Optionally, a first portion of the first surface comprising the adhesive does not overlap with a second portion of the first surface comprising the loop. Optionally, the second portion ranges from 10% to 70% of the first surface.

Optionally, the powder composition comprises granules with flowable characteristics, wherein the granules are of a size from 0.5 micron to 5 millimeters, and wherein the powder composition has a moisture content between 1% and 20%.

Optionally, an amount of the powder composition positioned in the recess is between 50 milligrams (mg) and 3000 mg.

Optionally, the powder composition comprises at least one of an anti-infective powder, an anti-fungal powder, an anesthetic powder, an analgesic powder, an anti-allergic powder, or a hemostat powder.

Optionally, the release film comprises at least one of polyethylene (PE), polyester (PET), polypropylene (PP), polyvinyl chloride (PVC), polytetrafluoroethylene (PTFE), wood pulp paper, synthetic paper, or glass fiber paper.

The present specification also discloses a delivery system for delivery of materials to a tissue surface, comprising: a powder composition; a loop defining a wall, wherein the loop has a first top surface and a second bottom surface; a first material continuously covering the first top surface; a second material continuously covering the second bottom surface, wherein the loop, the first material and the second material form an enclosure, and wherein the second material is a water-soluble membrane; a powder composition positioned inside the enclosure; and a release film adapted to cover the second material, wherein the release film is configured to be removed to place the second material in contact with a wound on the tissue surface and wherein the water-soluble membrane is adapted to dissolve in order to deliver the powder composition from the enclosure to the wound.

Optionally, the enclosure has a depth ranging from 0.1 mm to 20 mm.

Optionally, the first material has a thickness ranging from 0.15 mm to 0.3 mm and the first material comprises one or more of silicone gel, polyurethane (PU), polyethylene (PE), polyphenylene ether (PPE), polyvinyl chloride (PVC), cotton, non-woven fiber, synthetic fiber, clastic fabric, a waterproof material, or an elastic material.

Optionally, the loop is formed by one or more of a silicone gel, a foam, or cotton and the loop is at least one of a circular shape, an oval shape, a rectangular shape, a square shape or a circular ring with an inner diameter of 25 mm.

The present specification also discloses a delivery system for delivery of materials to a tissue surface, comprising: a powder composition; a loop forming a closed wall, the loop comprising a first top surface and a second bottom surface, wherein the powder composition is contained inside the wall; a first material continuously covering the first top surface including the powder composition, wherein the first material is configured to contain the powder composition within the closed wall of the loop from a top side of the loop; a second material continuously covering the second bottom surface including the powder composition, wherein the second material is configured to contain the powder composition within the closed wall of the loop from a bottom side of the loop, and wherein the second material comprises a removable cotton surface; and a release film adapted to cover the second material, wherein the release film is configured to be removed to place second material on a wound on the tissue surface, wherein the second material is pulled to rub the second material against the wound and the tissue surface for delivering the powder composition from the loop when the second material is removed.

Optionally, the cotton surface comprises a portion of the removable second material.

The present specification also discloses a method of treating a tissue surface of an organism having a wound therein comprising: removing a release film adhered to a bottom surface of a substrate, wherein the bottom surface of the substrate is covered by an adhesive and wherein a central portion of the bottom surface of the substrate comprises an enclosure containing a powder composition, wherein a bottom surface of the closed loop is covered by a water-soluble membrane adapted to adhere to at least a portion of the bottom surface of the substrate surrounding the closed loop, and a top surface of the closed loop is covered by the bottom surface of the substrate not containing the adhesive; adhering the bottom surface of the substrate with the adhesive to the tissue surface so that the wound is centrally positioned within the closed loop and the water-soluble membrane is in contact with the tissue surface; and allowing the water-soluble membrane to dissolve to release the powder composition on the wound.

The present specification also discloses a method of treating a tissue surface of an organism having a wound therein comprising: removing a release film adhering to a bottom surface of a closed loop, wherein the bottom surface of the closed loop is covered by an adhesive and wherein a central portion of the bottom surface of the closed loop contains a powder composition, wherein a bottom surface of the closed loop is covered partially by a water-soluble membrane adapted to adhere to at least a portion of the bottom surface of the closed loop surrounding the central portion and the water-soluble membrane covering the central portion of the closed loop to cover the powder composition along the bottom surface of the closed loop, and a top surface of the closed loop and the powder composition contained in said closed loop covered by a material; adhering the bottom surface of the closed loop with the adhesive to the tissue surface so that the wound is centrally position within the closed loop and the water-soluble membrane is in contact with the tissue surface; and allowing the water-soluble membrane to dissolve to release the powder composition on the wound.

The present specification also discloses a method of treating a tissue surface of an organism having a wound therein comprising: removing a release film to reveal a bottom surface of a removable cotton surface, wherein a top surface of the removable cotton surface is in contact with a bottom surface of a closed loop, wherein the bottom surface of the closed loop is covered by an adhesive and wherein a central portion of the closed loop contains a powder composition, and a top surface of the closed loop and the powder composition contained in said closed loop covered by a material; placing the bottom surface of the removable cotton surface over the tissue surface; pulling the removable cotton surface while rubbing the bottom surface of the removable cotton surface against the tissue surface, to remove the removable cotton surface; adhering the bottom surface of the closed loop with the adhesive to the tissue surface so that the wound is centrally position within the closed loop; and allowing the powder composition to be released on the wound.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A illustrates a bandage assembly of a dressing, in accordance with an embodiment of the present specification;

FIG. 1B is a flowchart detailing steps of an exemplary method for treating a tissue surface using the bandage assembly of FIG. 1A, in accordance with some embodiments of the present specification;

FIG. 2A is a side elevation view of a patch assembly, in accordance with an embodiment of the present specification;

FIG. 2B is a perspective view of an inner surface of the patch assembly of FIG. 2A without a release film, in accordance with an embodiment of the present specification;

FIG. 2C is a perspective view of an inner surface of the patch assembly of FIG. 2A with a release film, in accordance with an embodiment of the present specification;

FIG. 2D is an exploded diagram showing the exemplary embodiment of the patch assembly of FIG. 2A, in accordance with the present specification;

FIG. 3 is an illustration of an exemplary tab assembly used with the patch embodiments shown in FIGS. 2A, 2B, 2C, and 2D of the present specification;

FIG. 4A is a flowchart detailing an exemplary method of using a first embodiment of a patch assembly in accordance with the various embodiments of the present specification; and

FIG. 4B is a flowchart detailing an exemplary method of using a second embodiment of a patch assembly in accordance with the various embodiments of the present specification.

### DETAILED DESCRIPTION

According to some embodiments, the present specification describes biocompatible compositions and methods of use thereof to control bleeding and skin disease by achieving hemostasis and providing a healing function. In embodiments, hemostasis is achieved by application of a dressing comprising a medicament in a powder format, at the site of the wound. In some other embodiments, a biocompatible powder format composition is used to topically treat wounds or other designated sites on a surface of a human or animal body. In embodiments, the biocompatible composition is in a flowable, powder form. In embodiments, the biocompatible flowable powder composition is included within a dressing material that is protected by a water-dissolvable membrane for storage. The water-dissolvable membrane is used to retain the powder composition within the dressing material. The biocompatible composition can include, and is not limited to, drugs, anti-infectious agents, hemostatic materials, and compositions that promote healing. The flowable powder compositions used in various embodiments of the present specification can achieve hemostasis, anti-inflammatory functions, anti-infection functions, anesthetic functions, anti-immune functions, anti-allergy functions, cosmetic whitening treatment and generally promote healing.

In embodiments, the present specification provides blends of biocompatible compositions, in flowable powder form, contained within a dressing material. The presence of powder blends within the dressing material obviates the need for preparation and/or application time, which is required when using a powder composition separately from a dressing material. In embodiments, the water-soluble membrane provided to cover the flowable powder composition acts to seal the powder between the dressing material and the water-soluble membrane. Therefore, the water-soluble membrane serves to retain or restrain the flowable powder composition in the dressing, whereby the dressing includes, in some embodiments, a bandage or a patch.

In one embodiment, powder blends provide a longer shelf life, case of storage, and case of application of the dressing material compared to conventional compositions and dressing materials. The water-soluble membrane is configured to instantly dissolve with the powder composition upon application of the dressing material at the treatment site. It should be appreciated that in the systems and methods disclosed herein, multiple types of different powder compositions may be applied. In embodiments, the healing effect of the compositions described in the present specification can be achieved by application in situ (directly on the body site) without prior preparation or mixing.

The following definitions are provided to further describe various aspects of the preferred embodiments described in the present specification.

As used in various embodiments of the present specification, the degree of bleeding is defined qualitatively, wherein “mild” bleeding refers to blood oozing from a capillary; “moderate” bleeding is defined as when a wound site starts bleeding again immediately after wiping clean with a gauze; and “severe” bleeding is defined as that which cannot be controlled by wiping clean with gauze, and needs the use of a suction device. Embodiments of the present specification may be applied in cases where the bleeding is mild to moderate.

In embodiments, the degree of bleeding is determined by clinical definitions and are as follows:
With moderate bleeding, any of these may be true:
   The bleeding slows or stops with pressure but starts again if you remove the pressure.
   The blood may soak through a few bandages, but it is not fast or out of control.
With mild bleeding, any of these may be true:
   The bleeding stops on its own or with pressure.
   The bleeding stops or slows to an ooze or trickle after 15 minutes of pressure. It may ooze or trickle for up to 45 minutes.

The term “hemostat” or “hemostatic agent” refers to a substance used to control bleeding or promote hemostasis.

The term “sealant” refers to a substance used to block the passage of fluids through the surface or via openings.

The term “biodegradable” refers to the ability of the compositions of the present specification to be degraded or broken down (decomposed into smaller molecules owing to enzymatic action with the human body, which are then either absorbed by the body or eliminated via the kidney (urine).

The term “cohesive strength” refers to the ability of the compositions of the present invention to remain intact, i.e., not rupture, tear or crack, when subjected to physical stresses or environmental conditions. Cohesive strength is sometimes measured as a function of “burst strength”.

Furthermore, the following forms of measurements of different types of properties of different materials used in embodiments of the present specification are described below.

A durometer is used to measure the hardness of a material or substance. A relative indication of the hardness of a material may be shown on a scale representing the hardness from: very soft, soft, medium to hard. More specific measurements of hardness are sometimes derived using shore durometers (Shore 00 scale for very soft to soft materials, Shore A scale for soft to medium hard materials, and Shore D scale for hard materials).

Porosity is defined as a measure of the extent of voids or empty spaces in a material. The measure for porosity is often expressed as a value between 0 and 1 (or percentage between 0% and 100%), which represents a fraction of the volume of the voids over the total volume.

Viscosity is defined as the internal friction between layers of a fluid in motion and is indicative of the fluid's resistance to deformation under an applied force and is related to shear stress and velocity gradient. Viscosity is defined as a force multiplied by a time divided by an area and is represented in newton-seconds per square centimeter (Ns/cm²) or pascal-seconds (1 millipascal-seconds (mPa·s) is equal to 0.001 millipoise (mP).

Solubility is an expression of a degree to which a substance dissolves in a solvent; the solubility of the various compositions and/or materials used in the embodiments of the present specification is defined in terms of their ability to dissolve in water as the solvent.

Elasticity of a material represents its ability to return to its original shape and size after having been subjected to a distorting force. A highly elastic material returns to its original shape and size after a deforming force is applied and removed, indicating its resistance to deformity. A less elastic or a more plastic material tends to remain in a deformed state after removing the distorting force.

Tensile strength is a measure of the load-carrying capacity of a material until the material breaks or deforms. Tensile strength is often tested by stretching a material by applying a load, and measuring the maximum load that the material can support without breaking or fracturing.

The thickness of a material is measured as the distance between two opposing surfaces of the material.

It should be appreciated that the methods disclosed herein, specifically the application of one or more biocompatible powder format products to a wound or an otherwise affected site, is performed during the same, or during a single, operative procedure. The term “operative procedure” may be defined as a medical procedure involving a surface or an in vivo application of one or more tools to a living organism.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature, component of formulation described in association with a specific embodiment may be used and implemented with any other embodiment unless clearly indicated otherwise.

The present specification provides a number of biocompatible, biodegradable, hemostatic, anti-inflammatory, anti-infection, anesthetic, anti-immune agent, and anti-allergy agent powder compositions which may be used individually or in combination to achieve rapid and effective hemostasis or other desirable treatment. The compositions include flowable powder forms. The compositions of the present specification may be applied to humans as well as animals or mammals.

In embodiments, the present specification describes a dressing or bandage using the powder compositions described therein, whereby the dressing or bandage is an assembly comprising at least five components: a substrate with an adhesive, a groove within the substrate, a composition in powder form that is placed in the groove, a water-soluble membrane that is positioned above and used to restrain the powder composition between the substrate and the water-soluble membrane by adhering to the substrate, and a release film positioned above the water-soluble membrane.

### Substrate

A removable substrate with an adhesive layer on one side of the substrate, in the form of a bandage or a patch, or any other shape or thickness suitable for binding or wrapping an affected site on surface of a human/animal body, is provided. Conventional removable films, such as bandages, typically include a medicine-treated pad on its inner surface comprising an adhesive, that comes in direct contact with the affected site to consistently apply the medicine and facilitate healing. The removable substrate is placed directly on the affected site, such as a wound, a cut, an abrasion, a corn, an infection, or any other type of ailment, for protection and to facilitate the healing and treatment of the site. The adhesive is suitable for topical adhering on skin surface.

In embodiments where the removable substrate incorporates an elastic cloth as a base material, a pressure-sensitive adhesive, such as an acrylic adhesive, can be used. In embodiments where the removable substrate incorporates a silicone gel material as a base material, the silicone gel also provides the functions of a local adhesive. Different embodiments of the present specification can use any bio-safe adhesive suitable for application on human/animal tissue surface. The adhesive containing surface of the substrate is subsequently in contact with a body surface at the site of topical application of the powder composition, when the substrate is applied in the form of a dressing. The removable substrate is composed of one of polyethylene (PE), polyphenylene ether (PPE), polyvinyl chloride (PVC), cotton, non-woven fiber, synthetic fabric, an elastic fabric, water-proof material, elastic material, silicone gel material, among other types of known materials used for the purposes of topical dressing. In embodiments, the tolerance of the substrate material ranges between +1 mm.

### Groove in the Substrate

In embodiments, at least one groove is formed on a central portion of the surface of the substrate that includes the adhesive. In some embodiments, the groove is formed using silicone gel, foam, cotton or cotton sheets, or any other material in accordance with the embodiments of the present specification. In different embodiments, the depth of the groove is made from one or more of silicone gel, foam, cotton or cotton sheets, while one or more of the following materials form the bottom surface of the groove: polyurethane (PU), polyethylene (PE), PPE, polyvinyl chloride (PVC), cotton, non-woven fiber, synthetic fiber, elastic fabric, waterproof material, and elastic material. The adhesive on the substrate surface is, in some embodiments, applied around the groove so that there is no overlap of the adhesive with the area on the substrate that is covered by the groove. Therefore, the groove is positioned on the substrate surface in areas that do not comprise the adhesive. According to different embodiments, the depth of the groove ranges from 0.10 mm to 2 mm, and a percentage of the surface area of the substrate that is occupied by the groove ranges from 10% to 75%. In embodiments, the tolerance ranges between +1 mm. The groove forms a housing for a flowable powder composition.

### Powder Format Composition

In embodiments, the groove forms a housing for a dose of a powder composition, where the composition is made from a biocompatible material. The texture of the powder composition enables the housed quantity of the powder to flow smoothly to apply a target location of a wound. Topical powders, also known as powders for cutaneous application or powders for external use, are preparations consisting of solid, loose, dry particles of varying degrees of fineness. The powder composition of the present specification has flowable characteristics having particles or granules of a size that range from 0.5 micron to 5 millimeters (mm). In one embodiment, approximately 95% of the composition has granules of a size ranging from 1 micron to 500 microns.

In some embodiments, the powder compositions may be hydrophilic, not water-soluble, may form suspensions rather than solutions when exposed to water, or when exposed to water, may form a gel-like substance that promotes bleeding cessation. In some embodiments, the powder compositions that have hydrophilic properties are preferred and have a water absorption rate in a range of 1-100.

In embodiments, the moisture content of the powder composition is between 1% and 20%. Further, the loading range depends on the application of the powder composition and is typically in a range from 50-3000 mg.

The composition may include any compositions that are used for topical applications. Examples include, and are not limited to, anti-infectives such as bacitracin zinc and polymyxin B sulfate; or antifungals such as tolnaftate. Powder compositions offer a wide choice of ingredients suitable for various treatment objectives, which range from hemostat to anti-fungal, anti-infective and anesthetic, among other objectives. Some examples of powder format compositions that have anti-infection properties may include: cephalosporins, erythromycin, penicillin, ketoconazole, itraconazole, acyclovir, povidone-iodine, and chlorhexidine. Some examples of powder format compositions with analgesic properties may include: lidocaine, prilocaine, bupivacaine, morphine, and pethidine. Some examples of powder format compositions with anti-allergic properties may include: chlorpheniramine, loratadine, betamethasone, hydrocortisone, montelukast, and cetirizine. Some examples of powder format compositions that promote tissue healing may include: Epidermal Growth Factor (EGF), Fibroblast Growth Factor (FGF), Vascular Endothelial Growth Factor (VEGF), Interleukin-1 (IL-1), Transforming Growth Factor-beta (TGF-β), collagen granules, and adipose-derived stem cells. In embodiments, the powder format compositions with therapeutic functions can be used directly in their powder forms or states, or the therapeutic powder format compositions can be pre-mixed with non-functional or non-therapeutic powder compositions after forming a solution.

Further, examples of different types of bases that can be used to formulate powder compositions in accordance with the present specification include: starch-bases (hydroxyethyl starch (HES); CMS (carboxymethyl starch) and its sodium; Tert-amino-ethyl starch; HDMAEP-HES; The aldehyde group hydroxyethyl starch; Carbamyl ethylated carboxymethyl starch; The modified starch of dialdehyde schiff base; hydroxy propyl distarch phosphate; hydroxypropyl carboxymethyl starch; Polyhydroxy acetic acid grafted starch; Acrylamide is grafted with starch; sulfhydryl crosslinked starch; Sulfhydrated beta-cyclodextrin—starch composite microspheres; Dialdehyde starch; cross-linked amino starch); cellulose-bases (Carboxymethylcellulose (CMC); Hydroxyethyl Cellulose (HEC); hydroxy propyl cellulose; DCMC; Amino carboxymethyl cellulose; amino acid ethyl cellulose; Thiol modified carboxymethyl cellulose (CMC-SH); Amino hydroxyethyl cellulose; HPMC hydroxypropyl methyl cellulose); and Chitosan-based (Sulfhydryl carboxymethyl chitosan; DCMCS).

### Water-Soluble Film/Membrane (WSM)

In embodiments, a film that dissolves in contact with a fluid or a humid environment, is used to protect and seal the powder composition within the groove on the inner surface of the removable substrate. The WSM comprises a dissolvable film that protects the integrity of the powder composition during storage. The WSM ensures dryness and integrity of the powder composition by creating a barrier between the powder composition and the environment (which may contain humidity). The WSM is configured to break and dissolve when in contact with an amount of fluid such as water, blood, or any type of extrudate. The speed at which the WSM dissolves is directly proportional to the volume of fluid that it contacts upon application on a tissue at the site of treatment.

It is preferable for the WSM to quickly dissolve upon application, to permit the powder composition to directly contact with wound tissue. The dissolution of the protective film enables the powder composition to come into direct contact with the treatment site. Additionally, considering the clinical moisture environment of a wound and/or a disease tissue at the site of treatment, the WSM has the ability to fully/partially dissolve with small amount of blood or extrudate.

In embodiments, the WSM breaks or dissolves quickly when it is brought in contact with the wound or other affliction upon application of the dressing on a body surface. In some embodiments, a 1 square cm WSM is dissolved by 100 μl saline within 10 seconds. In some embodiments, a 1 square cm WSM is dissolved by 50 μl saline within 10 seconds. In some embodiments, a 1 square cm WSM is dissolved by 10 μl saline within 10 seconds. In some embodiments, a 1 square cm WSM is dissolved by 10 μl saline within 5 seconds. In some embodiments, the WSM is configured to dissolve and break in less than 30 seconds upon contact with a treatment site on a moisture wound of a human or animal. In an embodiment, the WSM dissolves and breaks in less than 5 seconds upon contact with a moisture wound. In an embodiment, the WSM dissolves and breaks in less than 5 seconds upon contact with a moist treatment site of a human or animal.

In embodiments, the WSM is composed of biocompatible and dissolvable elements including, but not limited to, starch or its derivatives. In some embodiments one or a combination of the following is used to prepare the water-soluble membrane: potato starch, modified starch, hydroxyethyl starch, carboxymethyl cellulose, microcrystalline cellulose, or any other type of cellulose. In one embodiment, the modified starch material according to the present specification includes starch modified physically, chemically, naturally, or enzymatically, and starch modified repeatedly with at least one of the above methods or a combination of two or more of the above methods.

The biocompatible material is also non-allergenic (hypo-allergenic), non-irritating, and non-cytotoxic. In embodiments, the water-soluble film has a tensile strength that is greater than or equal to 1N/15 mm. In addition, a thickness of the water-soluble film is in a range of 1 micrometer (μm) to 50 μm, and preferably within a range of 1 μm to 30 μm.

In embodiments, the WSM, which may also be referred to herein as ‘instant film’, has a smooth appearance with a uniform color and is void of bubbles. The presence of impurities and bubbles can be tested. In an exemplary method of testing, 4 square centimeters (cm²) of a sample of the manufactured WSM is taken and spread on a flat plate. The presence of spots or bubbles are observed from a distance of 20 cm from the sample. The numbers of spots and bubbles, if any, are counted. Impurities (pieces) with a diameter of ≤0.8 mm, and preferably of diameter≤0.5 mm, and of a quantity that is ≤25 pieces/m², and preferably ≤1 piece/cm², is considered to qualify for clinical applications of the present specification. Similarly, bubbles of diameter≤1 mm, and preferably ≤0.8 mm, and a quantity that is ≤25/mm², and preferably ≤1/cm², may be acceptable. Overall, if the diameter of impurities or bubbles on the 4 cm² sample meets the above requirements and the total number of impurities and bubbles is less than 4, the test sample is qualified for the clinical application in accordance with the present specification.

In embodiments, another method may be used to test the thickness of the WSM. An average of three points is taken along transverse and longitudinal directions, a point at each of the four corners of a rectangular or square WSM sample piece, and one point at the center. A thickness gauge, such as for example Labthink C640, is used to gauge the thickness of the 11 points. A mean and a standard deviation is calculated using the measurements of the 11 points to determine whether the thickness is within the qualifying range of 1 to 50 μm, and preferably in a range of 1 to 30 μm.

In addition, yet another method can be employed for testing the tensile strength of a sample piece of the WSM. Accordingly, 10 test specimens are each cut with a mold into pieces of length ranging from 30 to 80 mm, and a width of 15 mm. Standard test procedure known as ASTM F88/F88M-15 is used for this method. An intelligent electronic tensile testing machine is turned on and set to the option of “monitor key” to enable input of parameter setting of edge sealing strength. A distance between clamps is set to 50 mm, and the test speed to 25 mm/minute. Both sides of the intercepted test sample are clamped between upper and lower clamps of the machine. The test is initiated by activating the corresponding button on the machine. The test ends when the machine displays “Standby”. The value recorded at the end of the test is the edge sealing strength value. The upper and lower clamps are opened to retrieve the test sample. The qualifying limit for tensile strength, as may be determined by the above test, is ≥1N/15 mm.

Further, a test method can be used to determine the folding endurance of a sample of a WSM. In one exemplary method, a rectangular piece of WSM having dimensions of 1 cm length and 2 cm width, is repeatedly folded in the same position until the material is broken. At the point of breaking, the recorded number of times the sample of WSM is broken is its folding strength. In embodiments, a qualifying material for a WSM has a folding strength from 1 to 1000, and preferably greater than 200.

Yet another test can be performed to determine the dissolution time limit of a sample of WSM. In one case, the method is similar to that for determination of dissolution time limit of clotrimazole oral medicine film as described in the 20th Edition of Chinese Pharmacopocia. Accordingly, a WSM is cut into a size of 1 cm², 6 pieces of which are taken and used with two layers of stainless steel with an inner diameter of 2 mm mesh each. The pieces are wire clamped. Then, the clamped film is placed into the glass tube of the hanging basket for inspection. The film is added to 1000 ml of physiological saline is taken in a beaker at a temperature ranging from 36° C. to 38° C., moved up and down within a distance of 53 to 57 mm at a speed of 30-32 times/min. The disintegration time limit of the membrane is checked visually. Time spent collapse of the membrane is recorded, and an average value is calculated. The physiological saline remains clear after all the pieces of the WSM are dissolved. A qualifying material for a water-soluble membrane has a dissolution time limit of up to 25 seconds, and preferably of up to 10 seconds. Thus, the WSM has the ability to break and dissolve quickly to release the housed powder format composition as quickly as possible, when the WSM is brought in contact with the treatment site. The substrate and the grooves with adhesive do not have water-soluble properties, while the powder format composition, as discussed previously, has water-absorbing properties and may or may not dissolve in water.

An additional test can be conducted to ensure that the speed of penetration through a sample WSM qualifies and is within a range of 1-15 seconds, and preferably within a range of 0-5 seconds. For this purpose, a sample WSM material is laid flat on a gasket. The gasket may have a diameter of 3 cm and a height greater than 1 cm. The gasket has a smooth surface and may be made from any material. 1 ml of a physiological saline is drawn into a syringe (without needle), and quickly injected onto the surface of the WSM sample laid on the gasket. The speed is as fast as possible such that the impact of the saline on the WSM material does not exceed the diameter of the gasket. A stopwatch is used to record the time immediately from when the bolus is completed. The time taken for the physiological saline to penetrate the surface of the WSM is the penetration time and is an indication of the speed of penetration.

In embodiments, the WSM has a tolerance of +/−1 mm, along its length, width, and diameter. Also, in embodiments, the WSM has a moisture content of 4-20%.

### Release Film

A release film or liner is positioned above the WSM layer and is configured to be removed by a user prior to applying the dressing. Prior to application, the release film is pulled off from the surface of the substrate with the adhesive layer, to reveal the water-soluble membrane covering the powder format composition that is deposited inside the groove, and the surrounding adhesive of the substrate. A user can place the substrate while carefully positioning the central portion with the WSM directly over a treatment site or an affected area, while pressing the adhesive surface of the substrate against the surrounding tissue, to ensure that the substrate stays in its applied position. In embodiments, the release film or liner may include: polyethylene (PE), polyester (PET), polypropylene (PP), polyvinyl chloride (PVC), polytetrafluoroethylene (PTFE), wood pulp paper, synthetic paper (PET paper, PE paper), or glass fiber paper.

Table 1 provides a relative comparison of different properties of each component in the dressing assembly:

TABLE 1

| Dressing | | | | | | | |
|---|---|---|---|---|---|---|---|
| Assembly | | | Water | | Tensile | | Folding |
| Component | Durometer | Porosity | Solubility | Elasticity | Strength | Thickness | Endurance |
| Substrate | 1-500 | 5%-50% | — | Yes/No | ≥2.5N/15 | 0.05-0.4 | ≥1000 |
| | MPa | | | | mm | mm | |
| Groove | 1-30 | 20%-80% | — | Yes | ≥2.5N/15 | 0.2-0.8 | ≥500 |
| | MPa | | | | mm | mm | |
| Powder | — | — | Hydrophilic | — | — | — | — |
| Composition | | | (in some | | | | |
| | | | embodiments) | | | | |
| Water- | 0.1-20 | 1%-50% | Less than | No | ≥1N/15 | 1-50 | 1-1000 |
| Soluble | MPa | | 25 seconds | | mm | μm | |
| Membrane/ | | | | | | | |
| Film | | | | | | | |
| Release | — | — | — | No | ≥2.5N/15 | 0.1-0.6 | ≥1000 |
| film | | | | | mm | mm | |

### Bandage Embodiment

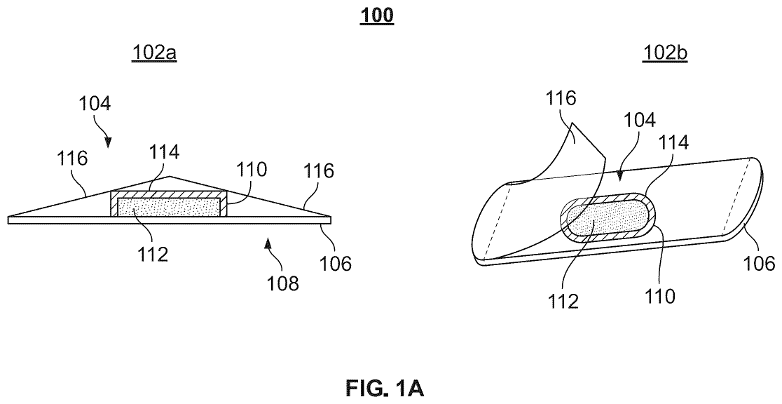

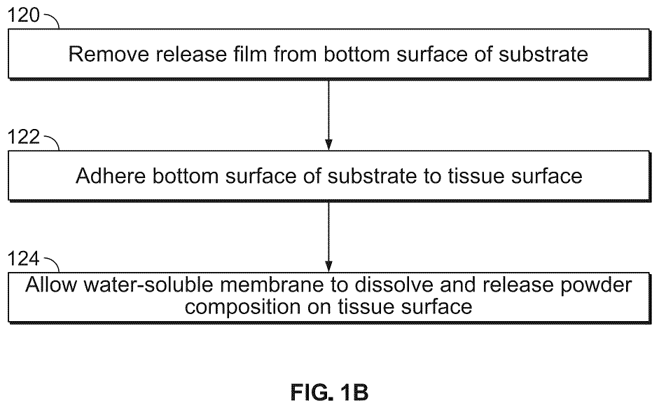

FIG. 1A illustrates an exemplary embodiment of a bandage assembly 100 of a dressing, in accordance with the present specification. In an embodiment, bandage assembly 100 has a length of approximately 90 mm and a width of approximately 30 mm. The figure shows a first side elevation view 102a and a second perspective view 102b of an inner surface 104 of bandage assembly 100. A removable substrate 106 of a rectangular shape has a first inner surface 104 and a second outer surface 108, opposite to inner surface 104. A person skilled in the art can appreciate that while the embodiment of substrate 106 illustrated and described herein is of a rectangular shape, substrate 106 can be of any other suitable shape in other embodiments, such as and not limited to square, oval, or circular. A length of the substrate 106 can range from 20 mm to 300 mm, and is approximately 90 mm in an embodiment, and its width can range from 10 mm to 200 mm, and is approximately 30 mm in an embodiment.

A closed loop structure, also herein referred to as a ‘groove’ 110 is configured on a central portion of substrate 106 such that groove 110 encloses an area ranging from 10% to 70% of inner surface 104 of substrate 106. The closed loop or groove 110 forms a wall of a specific thickness, enclosing said area within the loop. The thickness of the wall formed by groove 110 is in a range of 0.1 mm to 20 mm, and is approximately 7 mm in one embodiment. In one embodiment, groove 110 is rectangular in shape, with an outer length of its wall of approximately 25 mm and an inner length of the wall of approximately 18 mm; and an outer width of the wall of approximately 18 mm and an inner width of the wall of approximately 11 mm. Further in the same embodiment, groove 110 has a depth (height of the wall formed by the closed loop) ranging from 0.1 mm to 20 mm and is approximately 0.35 mm in one embodiment. Groove 110 is formed using one or more of: silicone gel, foam, cotton, or any other material in accordance with the embodiments of the present specification. Inner surface 104 of substrate 106 that surrounds an area outside of a space enclosed by groove 110 is coated with an adhesive that is appropriate for topical adhering. Exemplary embodiments of the adhesive may include pressure sensitive adhesives such as those that are acrylic-based, or silicone-gel-based adhesives. In embodiments, the area enclosed by groove 110 does not include the adhesive. The area enclosed by the wall of groove 110 provides a housing for a flowable powder composition 112, a dose of which is filled inside the housing against inner surface 104 of substrate 106.

While powder composition 112 occupies an inner side within the housing formed by the wall of groove 110 and inner surface of substrate 106, a water-soluble membrane 114 covers the housing formed by groove 110 from the other side, thereby completely enclosing powder composition 112. Water-soluble membrane 114 stretches over the wall formed by groove 110, covering the area enclosed therein. Water-soluble membrane 114 is made from one of: a starch film, a cellulose film, a polyester film, a composite film, or any other type of film described above with embodiments of water-soluble membrane 114. In embodiments, the adhesive coating on inner surface 104 of substrate 106 is present around water-soluble membrane 114. Thus, the adhesive coating is applied in a manner that avoids the area covered by water-soluble membrane 114.

In its product form, embodiments of the bandage assembly 100 further includes a release paper or film 116 that covers the entirety of inner surface 104 over and above water-soluble membrane 114, therefore covering and protecting water-soluble membrane 114 and the adhesive coating on inner surface 104 of substrate 106. FIG. 1B illustrates an exemplary method of use of bandage assembly 100. At step 120, a user first removes the innermost layer of release paper or film 116 of substrate 106. By removing the release paper the inner surface 104 of substrate 106 is revealed, and comprises the adhesive layer or coating and centrally positioned groove 110 covered by water-soluble membrane 114. At step 122, the user adheres the inner surface 104 of substrate 106 on a tissue surface. Substrate 106 is positioned on the tissue surface so that a wound requiring treatment is centrally located under inner surface 104 and so that groove 110 is positioned directly above the wound. Additionally, substrate 106 is preferably pressed by the user on its outer surface 108 to enable the adhesive on inner surface 104 to bond with the tissue surface. Once substrate 106 adheres to the tissue surface with the wound centrally located and covered by groove 110, at step 124, water-soluble membrane 114 that is now in contact with the tissue surface, and preferably is in contact with the wound, dissolves as a result of contacting the fluids that may be present in the wound or on the tissue surface. As water-soluble membrane 114 dissolves, powder composition 112 is exposed to the wound and is released on the wound. The released powder composition 112 comprising a drug composition subsequently supports treatment of the wound. The wound may alternatively be an injury, an ulcer, a corn, a rash, or any other type of affliction.

Assembly 100 can be used for multiple applications for topical treatments. Bandage assembly 100 with a suitable powder composition 112 is selected based on the required treatment, which may range among: hemostatic, pro-healing, analgesic, anti-allergy, and anti-infection, among others.

### Patch Embodiment

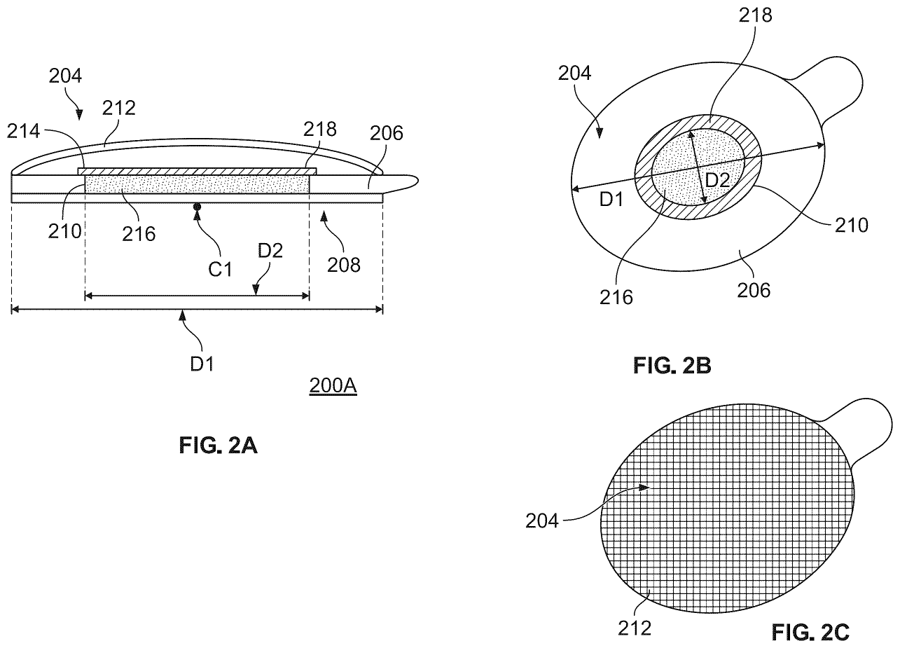

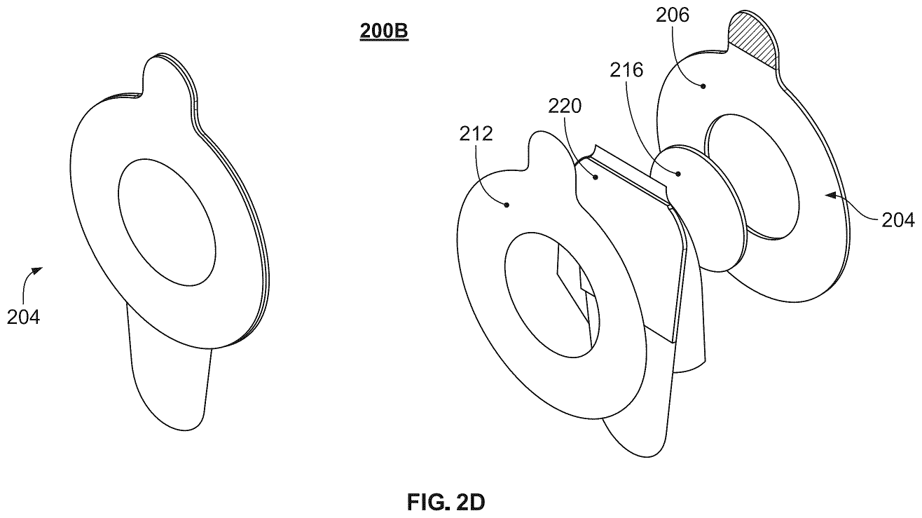

FIG. 2A is a side elevation view of an exemplary patch assembly 200A. FIG. 2B illustrates a perspective view of an inner surface 204 without a release film 212. FIG. 2C illustrates a perspective view of inner surface 204 with release film 212, in accordance with the present specification. FIGS. 2A, 2B and 2C showing a patch assembly are referred to simultaneously to describe a substrate 206. In some embodiments, substrate 206 is circular in shape, although in alternate embodiments, other shapes are also possible. An inner or bottom surface 204 of substrate 206 is opposite to an outer or top surface 208. Bottom surface 204 is configured to face a tissue surface of a subject at the time of application of patch assembly 200, while top surface 208 faces away from the tissue surface. Substrate 206 has a first diameter D1 and a center C1. Further, substrate 206 comprises a circular gap of a second diameter D2, where D2<D1, and wherein D1 and D2 share the center C1. In some embodiments, diameter D1 is in a range of 30 mm to 150 mm, and is 70 mm in one embodiment. Diameter D2 can be in a range of 10 mm to 120 mm, and is approximately 25 mm in one embodiment. The circular gap at the center of substrate 206 provides a ring or doughnut shape to inner surface 204.

A closed loop, also herein referred to as a circular ‘groove’, recess, or indent 210 is positioned such that it has a circumference with diameter D2 and defines the central circular gap in substrate 206. The closed loop, recess, indent or groove 110 forms a wall of a specific thickness, encircling and partially enclosing the area within the loop. The thickness of the wall formed by groove 210 is in a range of 0.1 mm to 20 mm, and is approximately 0.6 mm in one embodiment. In one embodiment, the indent or groove 210 is circular in shape, conforming with the shape of centrally positioned gap in substrate 206, with an outer diameter of approximately 25.6 mm and an inner diameter of approximately 25 mm. Inner surface 204 of substrate 206 that surrounds an area outside of the space enclosed by groove 210 is coated with an adhesive that is appropriate for topical adhering. Exemplary embodiments of the adhesive may include pressure sensitive adhesives such as those that are acrylic-based, or silicone-gel-based adhesives. Area enclosed by groove 210 does not include the adhesive. The area enclosed by the wall of groove 210 provides a housing for a flowable powder composition 216, a dose of which is filled inside the housing against a sheet of a first material 214 covering top surface of the wall formed by groove 210 extending towards inner surface 204 of substrate 206.

Further in the same embodiment, groove 210 has a depth (height of the wall formed by the closed loop) ranging from 0.1 mm to 20 mm, and is approximately 0.6 mm in one embodiment. Top surface of the wall formed by groove 210 faces substrate 206, while a bottom surface of the wall formed by groove 210 faces away from substrate 206. First material 214 stetches over top surface of groove 210 to support the housing formed within the wall of groove 210 for containing powder composition 216. Embodiments of first material 214 can include one or more of: polyurethane (PU), polyethylene (PE), PPE, polyvinyl chloride (PVC), cotton, non-woven fiber, synthetic fiber, elastic fabric, waterproof material, and elastic material. In some embodiments, first material 214 covers an entirety of inner surface 204 of substrate 210. In some embodiments, first material 214 is the same as substrate 206. A thickness of first material 214 (or substrate 206) is in a range from 0.15 mm to 0.3 mm. The wall of groove 210 is formed using one or more of: silicone gel, foam, cotton, or any other material in accordance with the embodiments of the present specification.

The depth of 0.6 mm enclosed by the wall of groove 210 provides a space to hold powder composition 216, where the powder composition can be in the form of a powder slug. The powder slug is held in its place, inside the wall of groove 210, with first material 214 covering the top surface of the housing and a second material such as a water-soluble membrane 218 providing a bottom cover to the housing. In the above embodiment where the powder slug has a diameter of approximately 25 mm (conforming with inner diameter of groove 210) and a depth of approximately 0.6 mm (conforming with depth of the wall formed by groove 210), approximately 500 milligrams (mg) of flowable powder composition 216 is housed within groove 210. In various embodiments shapes and sizes of groove 206 may utilize a combination of dimensions to enable housing different doses of flowable powder composition 216 to be used for topical treatment applications.

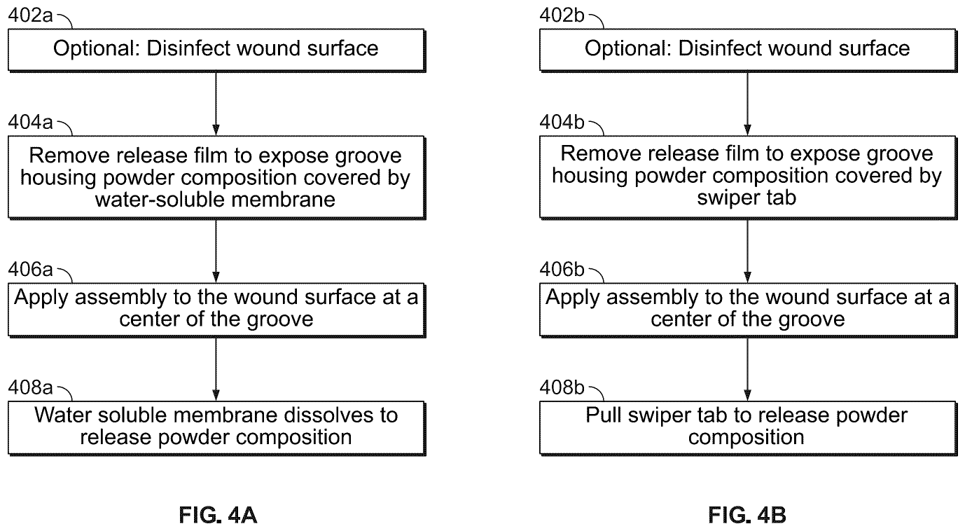

FIG. 4A is a flowchart detailing steps of an exemplary method for using patch assembly 200A. At step 402a, a tissue surface comprising an area requiring treatment, such as for example a wound, is optionally disinfected. The tissue surface can be disinfected using rubbing alcohol, water, saline, or any other disinfecting medium. At step 404a, release film 212 adhering to inner or bottom surface 204 of substrate 206 is removed to expose water-soluble membrane 218. At step 406a, assembly 200A is applied (with the exception of release film 212) to the tissue surface such that a wound or an area requiring treatment is at a center of the ring formed by groove 210. Substrate 206 is pressed to enable adherence of substrate 206 to the tissue surface with the help of the adhesive coating surrounding groove 210. At step 408a, water-soluble membrane 218 comes in contact with the area of treatment, and as a result water-soluble membrane 218 dissolves to release powder composition 216 housed inside the wall of the closed loop formed by groove 210.

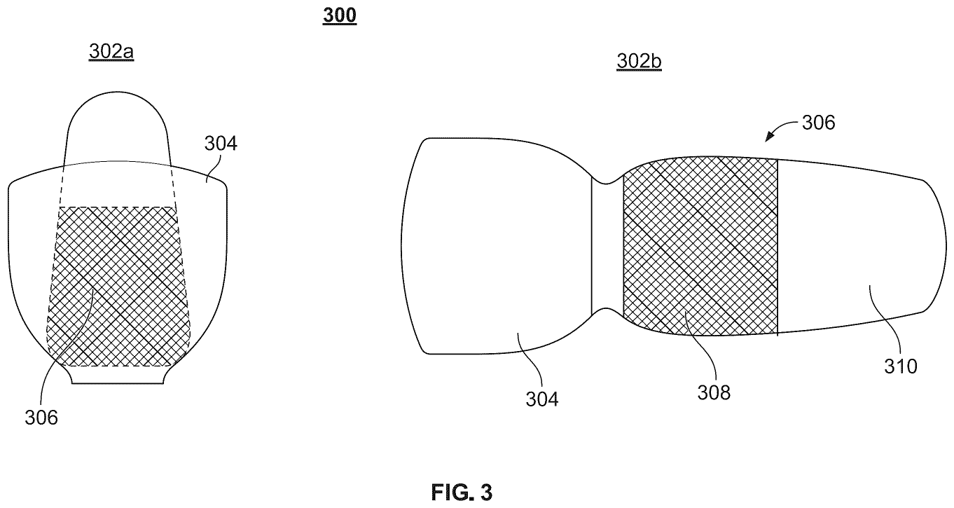

FIG. 2D illustrates another patch embodiment 200D in which water-soluble membrane 218 of FIGS. 2A and 2B is replaced with a removable cotton surface 220. The removable cotton surface 220 can also be referred to as ‘swiper tab’ since it provides the function of swiping the tissue surface comprising a wound or an area of treatment, where the swiping action is performed by pressing inner surface 204 of substrate 206 (after removing release film 212) against the tissue surface, and then pulling out cotton surface 220, which is initially sandwiched between inner surface 204 and the tissue surface, thereby allowing cotton surface 220 to rub against the tissue surface and the area of treatment. Details of cotton surface 220 are provided with reference to FIG. 3. An innermost layer of a release liner or film 212 is provided on bottom surface of cotton surface 220, which is opposite to the surface that faces powder composition 216. Powder composition 216 is preferably compressed and housed in the form of a powder slug. In embodiments, dimensions of release film 212 are similar to those of substrate 206. Release film 212 covers the entirety of inner surface 204 of substrate 206. In the embodiments, such as those illustrated in FIG. 2D, function of water-soluble membrane 218 is performed by cotton surface 220. Cotton surface 220 provides a means to shield and protect the powder slug comprising powder composition 216.

FIG. 4B is a flowchart detailing steps of an exemplary method of use of assembly 200D. At step 402b, a tissue surface comprising an area requiring treatment, such as for example a wound, is optionally disinfected. The tissue surface can be disinfected using rubbing alcohol, water, saline, or any other disinfecting medium. At step 404b, release film 212 is removed to expose cotton surface 220. At step 406b, assembly 200D is applied (with the exception of release film 212) to the tissue surface such that a wound or an area requiring treatment is at a center of the closed loop formed by wall of groove 210. Cotton surface 220 is now sandwiched between the tissue surface and substrate 206. At step 408b, a portion of cotton surface 220 that is configured to extend outwards from substrate 206 is held and pulled. The pulling action brushes cotton surface 220 against the area of treatment on the tissue surface, resulting in removal of any fluids or dirt such as blood that may be present on the tissue surface. By continuing to pull on cotton surface 220, it is completely removed from assembly 200D and releases powder composition 216.

The area requiring treatment on the tissue surface, may be a wound, an injury, an ulcer, a corn, a rash, or any other type of affliction. Assembly 200A or 200D can be used for multiple applications for topical treatments. Patch assembly 200A/200D with a suitable powder composition and its dose within powder composition 216 is selected based on the required treatment, which may range among: hemostatic, pro-healing, analgesic, anti-allergy, and anti-infection, among others.

FIG. 3 illustrates an exemplary swiper tab assembly 300 (cotton surface 220 of FIG. 2D) used with the patch embodiments 200D of FIG. 2D of the present specification. Assembly 300 is made of a first portion 304 and a second portion 306, which are continually attached to each other. A first view 302a shows assembly 300 in a folded configuration, where second portion 306 is folded to cover inner surface of first portion 304; and a second view 302b illustrates a straightened configuration of assembly 300 showing the surfaces of portions 304 and 306 that face each other in the first folded configuration. Patch assembly 200D is available with tab assembly 300 in the first configuration, and positioned upon the bottom or inner surface of groove 210 so that the powder slug comprising powder composition 216 is shielded within groove 210 by tab assembly 300 on the bottom side, and on the top side by first material 214 covering groove 210. Upon application of patch assembly 200D, after removing removable film 212 and positioning groove 210 around the area requiring treatment, tab assembly 300 is brought into its second configuration by pulling of second portion 306.

In some embodiments, first portion 304 is pentagon shaped, with four curving sides and the fifth side is attached to second portion 306. A length of portion 304 can range from 40 to 50 mm, and its width can range from 25 to 40 mm. Portion 304 is made from a material such as and not limited to PET and PE. In embodiments, portion 304 is in contact with powder composition 216 until tab assembly 300 is pulled and removed to bring powder composition 216 in direct contact with the tissue surface. Portion 306 continually extends from portion 304 in an elongated bottle-shape with a length ranging from 20 to 35 mm. A fraction ranging from 50% or more, of inner surface of portion 306, which is proximal to portion 304, is comprised of an absorbent cotton pad 308. Cotton pad 308 is made from an absorbent material such as cotton that is also soft and can assist in removing dirt, blood, and otherwise cleaning the wound. Upon application of patch assembly 200D, tab assembly 300 is held from the remaining extending surface 310 of second portion 306 which is not covered by cotton pad 308, resulting in rubbing of cotton pad 308 surface against the tissue surface and cleaning of the area requiring treatment. Surface 310 is held and pulled during the rubbing of cotton pad 308 up to the pulling out and removal of the entire tab assembly 300 from patch assembly 200D.

The above examples are merely illustrative of the many applications of the systems and methods of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A bandage adapted to deliver a powder composition to tissue on a body surface, comprising:
a removable substrate comprising an adhesive on a first surface adapted to face the tissue;
a loop having a thickness and defining a recess characterized by a depth, wherein the recess is positioned centrally on said first surface;
the powder composition positioned in the recess;
a water-soluble membrane adapted to cover the recess and the powder composition positioned in the recess, wherein the water-soluble membrane covers the loop and is adapted to adhere to the first surface of the removable substrate; and
a release film adapted to cover the water-soluble membrane and adapted to adhere to the first surface of the removable substrate.

2. The bandage of claim 1, wherein the water-soluble membrane is adapted to dissolve in less than 30 seconds after removing the release film and adhering the removable substrate to the tissue.

3. The bandage of claim 1, wherein the water-soluble membrane is adapted to dissolve in less than 10 seconds when the water-soluble membrane is in contact with at least 0.05 milliliters of a fluid.

4. The bandage of claim 1, wherein the water-soluble membrane has a tensile strength of at least 1N/15 mm.

5. The bandage of claim 1, wherein the water-soluble membrane is at least one of non-allergic, non-irritating, or non-cytotoxic.

6. The bandage of claim 1, wherein the water-soluble membrane has a thickness between 1 μm and 500 μm.

7. The bandage of claim 1, wherein the water-soluble membrane comprises at least one of potato starch, modified starch, hydroxyethyl starch, carboxymethyl cellulose, or microcrystalline cellulose.

8. The bandage of claim 1, wherein the water-soluble membrane has a folding endurance between 10 and 1000.

9. The bandage of claim 1 wherein the adhesive is at least one of a pressure sensitive adhesive or an acrylic based adhesive.

10. The bandage of claim 1, wherein the removable substrate comprises at least one of polyethylene (PE), polyphenylene ether (PPE), polyvinyl chloride (PVC), cotton, non-woven fiber, synthetic fabric, an elastic fabric, a water-proof material, or a silicone gel material.

11. The bandage of claim 1, wherein the loop comprises at least one of a silicone gel, foam, or cotton and is of a rectangular shape, a square shape, an oval shape, or a circular shape.

12. The bandage of claim 1 wherein a first portion of the first surface comprising the adhesive does not overlap with a second portion of the first surface comprising the loop.

13. The bandage of claim 12, wherein the second portion ranges from 10% to 70% of the first surface.

14. The bandage of claim 1, wherein the powder composition comprises granules with flowable characteristics, wherein the granules are of a size from 0.5 micron to 5 millimeters, and wherein the powder composition has a moisture content between 1% and 20%.

15. The bandage of claim 1, wherein an amount of the powder composition positioned in the recess is between 50 milligrams (mg) and 3000 mg.

16. The bandage of claim 1, wherein the powder composition comprises at least one of an anti-infective powder, an anti-fungal powder, an anesthetic powder, an analgesic powder, an anti-allergic powder, or a hemostat powder.

17. The bandage of claim 1, wherein the release film comprises at least one of polyethylene (PE), polyester (PET), polypropylene (PP), polyvinyl chloride (PVC), polytetrafluoroethylene (PTFE), wood pulp paper, synthetic paper, or glass fiber paper.

18. A delivery system for delivery of materials to a tissue surface, comprising:
a powder composition;
a loop defining a wall, wherein the loop has a first top surface and a second bottom surface;
a first material continuously covering the first top surface;
a second material continuously covering the second bottom surface, wherein the loop, the first material and the second material form an enclosure, and wherein the second material is a water-soluble membrane;
a powder composition positioned inside the enclosure; and
a release film adapted to cover the second material, wherein the release film is configured to be removed to place the second material in contact with a wound on the tissue surface and wherein the water-soluble membrane is adapted to dissolve in order to deliver the powder composition from the enclosure to the wound.

19. The delivery system of claim 18, wherein the enclosure has a depth ranging from 0.1 mm to 20 mm.

20. The delivery system of claim 18, wherein the first material has a thickness ranging from 0.15 mm to 0.3 mm and wherein the first material comprises one or more of silicone gel, polyurethane (PU), polyethylene (PE), polyphenylene ether (PPE), polyvinyl chloride (PVC), cotton, non-woven fiber, synthetic fiber, elastic fabric, a waterproof material, or an elastic material.

21. The delivery system of claim 18, wherein the loop is formed by one or more of a silicone gel, a foam, or cotton and wherein the loop is at least one of a circular shape, an oval shape, a rectangular shape, a square shape or a circular ring with an inner diameter of 25 mm.
